(19) United States
(12) Patent Application Publication
(10) Pub. No.: US 2025/0116582 A1
(43) Pub. Date: Apr. 10, 2025

(54) SAMPLE PROCESSING APPARATUS, SAMPLE PROCESSING SYSTEM, AND CARTRIDGE

(71) Applicants: CANON MEDICAL SYSTEMS CORPORATION, Otawara-shi (JP); Canon Kabushiki Kaisha, Tokyo (JP)

(72) Inventors: Tomoki FUJITO, Nasushiobara (JP); Tsutomu MIKI, Komae (JP); Yuhi HIGUCHI, Shinagawa (JP); Shinya SUGIYAMA, Nasushiobara (JP); Hirotoshi TAHARA, Nasushiobara (JP); Kazutaka YANAGITA, Yokohama (JP); Tetsuya SAKAI, Ryugasaki (JP); Akane KUBOYAMA, Kawasaki (JP)

(73) Assignees: CANON MEDICAL SYSTEMS CORPORATION, Otawara-shi (JP); Canon Kabushiki Kaisha, Tokyo (JP)

(21) Appl. No.: 18/904,888

(22) Filed: Oct. 2, 2024

(30) Foreign Application Priority Data
Oct. 6, 2023 (JP) 2023-174362

(51) Int. Cl. G01N 1/34

(52) U.S. Cl. CPC G01N 1/34

(57) ABSTRACT

A sample processing apparatus according to the present embodiment includes an apparatus body. In the apparatus body, a cartridge for internally performing processing on a cell is installed, and the apparatus body controls liquid feeding in the cartridge and/or adjusts an environment in the cartridge. The apparatus body includes processing circuitry configured to detect an inclination of the cartridge with respect to a horizontal plane and/or the apparatus body.

### CROSS-REFERENCE TO RELATED APPLICATIONS

This application is based upon and claims the benefit of priority from Japanese Patent Application No. 2023-174362, filed on Oct. 6, 2023; the entire contents of which are incorporated herein by reference.

### FIELD

Embodiments described herein relate generally to a sample processing apparatus, a sample processing system, and a cartridge.

### BACKGROUND

Hitherto, production of induced pluripotent stem cells (hereinafter, referred to as iPS cells) has been manually carried out by a skilled worker with equipment such as a safety cabinet. The equipment such as a safety cabinet is expensive, and labor costs of workers are also required, which makes the production of the iPS cells very costly.

### BRIEF DESCRIPTION OF THE DRAWINGS

FIG. 1 is a diagram illustrating an example of an appearance of a sample processing system according to an embodiment;

FIG. 2 is a diagram illustrating an example of a configuration of a sample processing apparatus according to the embodiment;

FIG. 3 is a diagram illustrating an example of an internal configuration of a cartridge when viewed from an X direction according to the embodiment;

FIG. 4 is a view illustrating an example of an appearance of the cartridge according to the embodiment;

FIG. 5 is a diagram illustrating an example of a positional relationship between a level and a reading apparatus that reads the level according to the embodiment;

FIG. 6 is a diagram illustrating an example of movement MDM of the cartridge by a conveyance apparatus according to the embodiment;

FIG. 7 is a flowchart illustrating an example of a procedure of cartridge inclination detection processing according to the embodiment;

FIG. 8 is a diagram illustrating an example of a positional relationship between the level and a reading apparatus 631 that reads the level according to a first modification of the embodiment;

FIG. 9 is a diagram illustrating an example of a positional relationship between a contact apparatus and the cartridge according to a second modification of the embodiment;

FIG. 10 is a view illustrating an example of a positional relationship between an arm and the cartridge at two positions according to a third modification of the embodiment;

FIG. 11 is a diagram illustrating an example of a positional relationship between a plurality of sensors and the cartridge according to a fourth modification of the embodiment; and

FIG. 12 is a flowchart illustrating an example of a procedure of cartridge inclination detection processing according to the fourth modification of the embodiment.

### DETAILED DESCRIPTION

As a method for reducing cost required for producing an induced pluripotent stem cell (iPS cell), for example, it is conceivable to adopt a apparatus configuration in which a cartridge including a flow path for processing cell suspension is set in a apparatus having a liquid feeding function to execute a liquid feeding process. However, the present inventors have found that when the cartridge is inclined, a filter for collecting a target cell and/or a culture container and the like in the cartridge are inclined, as a result of which the processing of the cell suspension cannot be appropriately performed in some cases.

A sample processing apparatus according to an embodiment includes an apparatus body. In the apparatus body, a cartridge for internally performing processing on a cell is installed, and the apparatus body controls liquid feeding in the cartridge and/or adjusts an environment in the cartridge. The apparatus body includes processing circuitry configured to detect an inclination of the cartridge with respect to a horizontal plane and/or the apparatus body.

Hereinafter, the sample processing apparatus, a sample processing system, and the cartridge will be described with reference to the drawings. In the following embodiment, portions denoted by the same reference numerals perform similar operations, and an overlapping description will be omitted as appropriate. In addition, contents described in one embodiment are similarly applied to modifications, application examples, and the like in principle.

### Embodiment

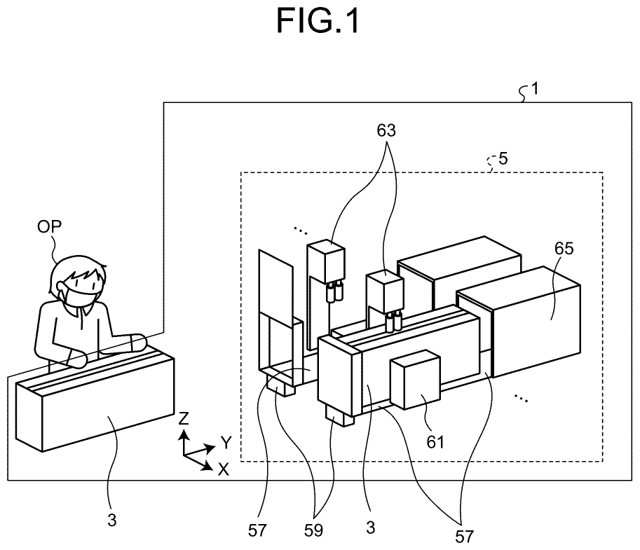

FIG. 1 is a diagram illustrating an example of an appearance of a sample processing system 1 according to the present embodiment. As illustrated in FIG. 1, the sample processing system 1 includes a cartridge 3 and a sample processing apparatus 5. The cartridge 3 is inserted into the sample processing apparatus 5 by an operator (user) OP. The cartridge 3 is filled with a sample and a reagent in advance by the operator OP before being inserted into the sample processing apparatus 5.

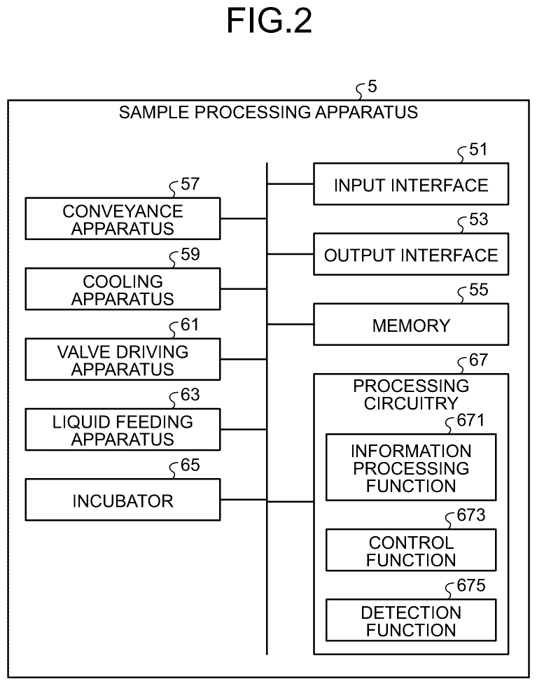

FIG. 2 is a diagram illustrating an example of a configuration of the sample processing apparatus 5. As illustrated in FIG. 2, the sample processing apparatus 5 includes an input interface 51, an output interface 53, a memory 55, a conveyance apparatus 57, a cooling apparatus 59, a valve driving apparatus 61, a liquid feeding apparatus 63, an incubator 65, and processing circuitry 67. In an apparatus body of the sample processing apparatus 5, the cartridge 3 for internally performing processing on a cell is installed. As a result, the apparatus body controls liquid feeding in the cartridge and/or adjusts an environment in the cartridge.

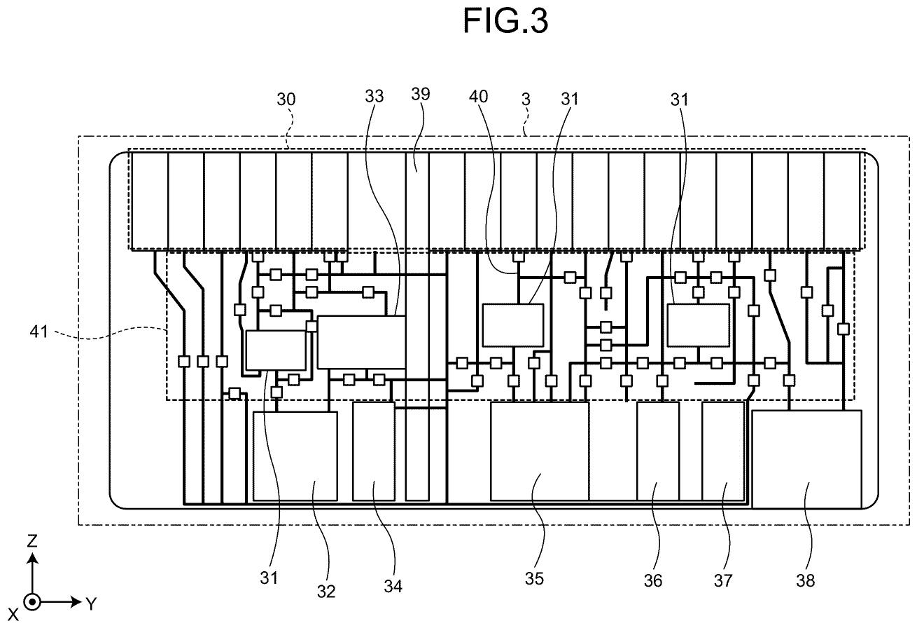

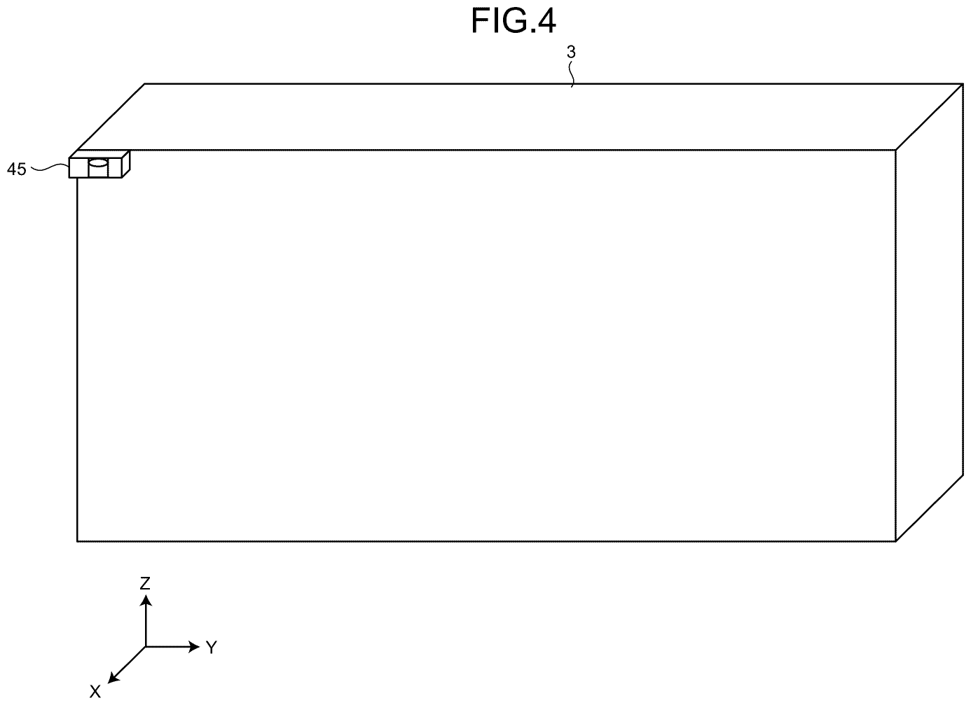

Hereinafter, the cartridge 3 will be described with reference to FIGS. 3 and 4. FIG. 3 is a diagram illustrating an example of an internal configuration of the cartridge 3 when viewed from an X direction. FIG. 4 is a diagram illustrating an example of an appearance of the cartridge 3. The cartridge 3 internally performs processing on a cell that is the sample. Specifically, the cartridge 3 performs processing on the cell by various operations performed by the sample processing apparatus 5.

As illustrated in FIG. 3, the cartridge 3 includes, for example, a sample container, a reagent container, a plurality of storage containers 30, a filter 31 for collecting a target cell, a waste liquid container 32, a spiral flow path 33, a tapered container 34, an expansion culture container 35, an infection container 36, a pre-culture container 37, a culture container 38 for cell culture, a heat insulating material 39, a plurality of tubes 40, and a plurality of valves 41. As illustrated in FIG. 4, a level 45 is attached to the cartridge 3.

In the cartridge 3, the sample container and the reagent container are arranged on a back side of the plurality of storage containers 30 illustrated in FIG. 3. The sample container holds a sample to be sucked. The sample is, for example, human blood. Here, the sample container corresponds to a blood pack. The sample is not limited to blood, and may be another turbid liquid. The reagent container holds a reagent to be sucked. In a case where the sample processing apparatus 5 produces the induced pluripotent stem cell (hereinafter, referred to as iPS cell), the reagent is, for example, a reagent necessary for producing the iPS cell. A known reagent can be applied as the reagent necessary for producing the iPS cell, and thus a description thereof is omitted.

For example, physiological saline is stored in the plurality of storage containers 30. For example, phosphate-buffered saline (PBS) may be used as the physiological saline. The storage container 30 stores the sample sucked from the sample container by the liquid feeding apparatus 63 described below. Among the plurality of storage containers 30, a storage container that is different from the storage container in which the sample is stored stores the sample sucked from the reagent container by the liquid feeding apparatus 63. Another storage container among the plurality of storage containers 30 stores a residue remaining in the filter 31 collected by backwashing of the filter 31. At this time, the another storage container 30 may be referred to as a collection container.

The filter 31 filters out a fine particle in the sample that is suspension. For example, the filter 31 collects a target cell for cell culture performed in the culture container 38. The fine particle as the target cell is, for example, a red blood cell or a white blood cell. The waste liquid container 32 is, for example, a container for collecting the suspension filtered by the filter 31. A description of the spiral flow path 33, the tapered container 34, the expansion culture container 35, the infection container 36, and the pre-culture container 37 will be omitted.

The sample and the reagent are mixed in the culture container 38. The culture container 38 may be referred to as a mixing container in which the sample and the reagent are mixed. For example, the samples subjected to various types of processing and the reagents are mixed in the culture container 38. The culture container 38 is a container used for cell culture. A content (the target cell related to cell culture) in the culture container 38 is cultured by the incubator 65 described below.

As illustrated in FIG. 3, the heat insulating material 39 is arranged between the tapered container 34 and the expansion culture container 35. By the heat insulating material 39, for example, a temperature and the like of the culture container 38 and the expansion culture container 35 in the cartridge 3 inserted into the incubator 65 are kept constant.

As illustrated in FIG. 3, the plurality of tubes 40 connect, for example, the sample container, the reagent container, the plurality of storage containers 30, the filter 31, the waste liquid container 32, the spiral flow path 33, the tapered container 34, the expansion culture container 35, the infection container 36, the pre-culture container 37, the culture container 38, and the like. As illustrated in FIG. 3, a flow path formed by the plurality of tubes 40 is implemented as a closed system (hereinafter, referred to as a closed system flow path).

The plurality of valves 41 are provided in the plurality of tubes 40. In other words, the plurality of tubes 40 are respectively provided with the plurality of valves 41. Each of the plurality of valves 41 uses a magnet or a magnetic body as a member for opening and closing the tube 40. Each of the plurality of valves 41 switches a flow path in the closed system flow path via a cover (wall) of the cartridge 3 that is parallel to a YZ plane under the control of a control function 673 of the processing circuitry 67 by the valve driving apparatus 61 described below.

As illustrated in FIG. 4, the cartridge 3 includes the level 45. The level 45 is attached to, for example, a housing forming an exterior of the cartridge 3 such that a bottom surface or an upper surface of the cartridge 3 indicates the horizontal plane. The level 45 measures a horizontal state of the cartridge 3. The horizontal state is, for example, a numerical value reflecting the degree of horizontality of the cartridge 3, a gradient of the cartridge 3, or a position of a bubble in a bubble tube of the level 45. Since a known level is applicable as the level 45, a description thereof will be omitted. An angle meter that detects the inclination of the cartridge 3 with respect to the horizontal plane may be used as the level 45. An attachment position of the level 45 is not limited to that illustrated in FIG. 4, and can be set at any position on the upper surface of the cartridge 3.

Hereinafter, the configuration of the sample processing apparatus 5 will be described with reference to FIGS. 1 and 2.

The input interface 51 receives various input operations from the operator OP who operates the sample processing apparatus 5, converts the received input operations into electric signals, and outputs the electric signals to the processing circuitry 67. For example, the input interface 51 receives a selection instruction, various conditions, and the like from the operator. As the input interface 51, for example, a mouse, a keyboard, a trackball, a switch, a button, a joystick, a touch pad, or a touch panel display can be appropriately used.

In the present embodiment, the input interface 51 is not limited to one including a physical operation component such as a mouse, a keyboard, a trackball, a switch, a button, a joystick, a touch pad, or a touch panel display. For example, electric signal processing circuitry configured to receive an electric signal corresponding to an input operation from an external input device provided separately from the device and output the electric signal to the processing circuitry 67 is also included in the example of the input interface 51. The input interface 51 may be implemented by a tablet terminal or the like that can wirelessly communicate with the body of the sample processing apparatus 5.

The output interface 53 includes, for example, a communication interface and a display. The output interface 53 may further include a speaker that outputs a sound to the operator OP. The communication interface performs, for example, data communication between the sample processing apparatus 5 and a network. A standard of communication between the communication interface and the network may be any standard.

The display displays various types of information. For example, the display outputs a graphical user interface (GUI) or the like for receiving various operations from the user. For example, a liquid crystal display (LCD), a cathode ray tube (CRT) display, an organic electro luminescence display (OELD), a plasma display, or any other display can be appropriately used as the display. In addition, the display may be a desktop type or may be implemented by a tablet terminal or the like that can wirelessly communicate with the sample processing apparatus 5.

The memory 55 is a storage device such as a hard disk drive (HDD), a solid state drive (SSD), or integrated circuitry storage device that stores various types of information. The memory 55 may be a driving device that reads and writes various types of information from and to a portable storage medium such as a compact disc (CD), a digital versatile disc (DVD), or a flash memory, a semiconductor memory element such as a random access memory (RAM), or the like, in addition to the HDD, the SSD, or the like.

The memory 55 stores, for example, various programs related to execution of an information acquisition function 671, the control function 673, a detection function 675, and the like. The memory 55 stores information regarding sample processing (hereinafter, referred to as sample processing information) acquired from the input interface 51 or the like by the information acquisition function 671. The sample processing information is information regarding processing of the sample, and is, for example, a suction amount of the sample from the sample container, a suction amount of the reagent sucked from the reagent container, an arrangement time when the culture container (mixing container) 38 is arranged in the incubator 65, and the like. The memory 55 stores the inclination of the cartridge 3 detected by the detection function 675.

The conveyance apparatus 57 moves the cartridge 3 to a position (hereinafter, referred to as a suction/discharge position) related to suction and discharge of the sample and the reagent under the control of the control function 673. The conveyance apparatus 57 moves the cartridge 3 from the suction/discharge position to the incubator 65 by the control function 673. The conveyance apparatus 57 moves the cartridge 3 from the incubator 65 to the suction/discharge position by the control function 673. The conveyance apparatus 57 moves the cartridge 3 from the sample processing apparatus 5 to the outside of the sample processing apparatus 5 by the control function 673. For example, the conveyance apparatus 57 on which the cartridge 3 can be placed conveys the cartridge 3 in a horizontal direction. The conveyance apparatus 57 corresponds to a conveyance unit.

When the cartridge 3 is inserted into the sample processing apparatus 5, the cooling apparatus 59 cools the cartridge 3 under the control of the control function 673. The cooling apparatus 59 is implemented by, for example, a Peltier element. The cooling apparatus 59 cools, for example, one side surface of the cartridge 3 that is in contact with the cooling apparatus 59 by the control function 673.

The valve driving apparatus 61 drives opening and closing of the valve 41 by magnetic interaction with the magnet or the magnetic body in the valve 41 under the control of the control function 673. That is, the valve driving apparatus 61 drives opening and closing of the valve 41 by magnetic interaction from the outside of the cartridge 3 through the cover of the cartridge 3. Specifically, the valve driving apparatus 61 includes a magnet (hereinafter, referred to as a second magnet) that attracts the magnet (hereinafter, referred to as a first magnet) or the magnetic body in the valve 41 via the cover of the cartridge 3, a movement support mechanism that supports the second magnet so as to be movable in the horizontal direction, and a power source that supplies power to the movement support mechanism. The valve driving apparatus 61 corresponds to a valve driving unit.

The movement support mechanism is implemented by, for example, an actuator that supports the second magnet at a distal end. For example, a known actuator such as a double-acting air cylinder, a single-acting air cylinder, or a hydraulic cylinder may be used as the movement support mechanism. The movement support mechanism moves the second magnet close to the cartridge 3 or moves the second magnet away from the cartridge 3 under the control of the control function 673. As a result, the first magnet or the magnetic body in the valve 41 moves, and opening and closing of the valve 41 is implemented.

The liquid feeding apparatus 63 includes a pressure pad (which may also be referred to as a pressurizing pad) movable in a vertical direction (Z direction) and a movement mechanism movable in a long-axis direction (Y direction) of the cartridge 3. The liquid feeding apparatus 63 is moved such that a pressure pad connection portion connected to the storage container 30 is positioned immediately below the pressure pad under the control of the control function 673. Next, the liquid feeding apparatus 63 lowers the pressure pad arranged at a distal end of the liquid feeding apparatus 63 so as to be in close contact with the pressure pad connection portion by the control function 673. Subsequently, the liquid feeding apparatus 63 sucks or compresses air in a pressure chamber via the pressure pad and the pressure pad connection portion by the control function 673. As a result, a pressure in the storage container is reduced or increased, and the sample, the reagent, or the like is sucked into or flown out through the tube 40. Thus, the liquid feeding apparatus 63 controls liquid feeding in the cartridge.

The liquid feeding apparatus 63 includes a reading apparatus that reads the level. For example, the reading apparatus is mounted on the liquid feeding apparatus 63 in the vicinity of a distal end of the pressure pad or the like. The reading apparatus reads, for example, the position of the bubble in the level by, for example, an optical method. The reading apparatus is not limited to an apparatus that implements optical reading, and may be implemented by, for example, a reader using an electromagnetic wave, a laser, or the like. Since a known reading apparatus can be applied as the reading apparatus, a description thereof will be omitted.

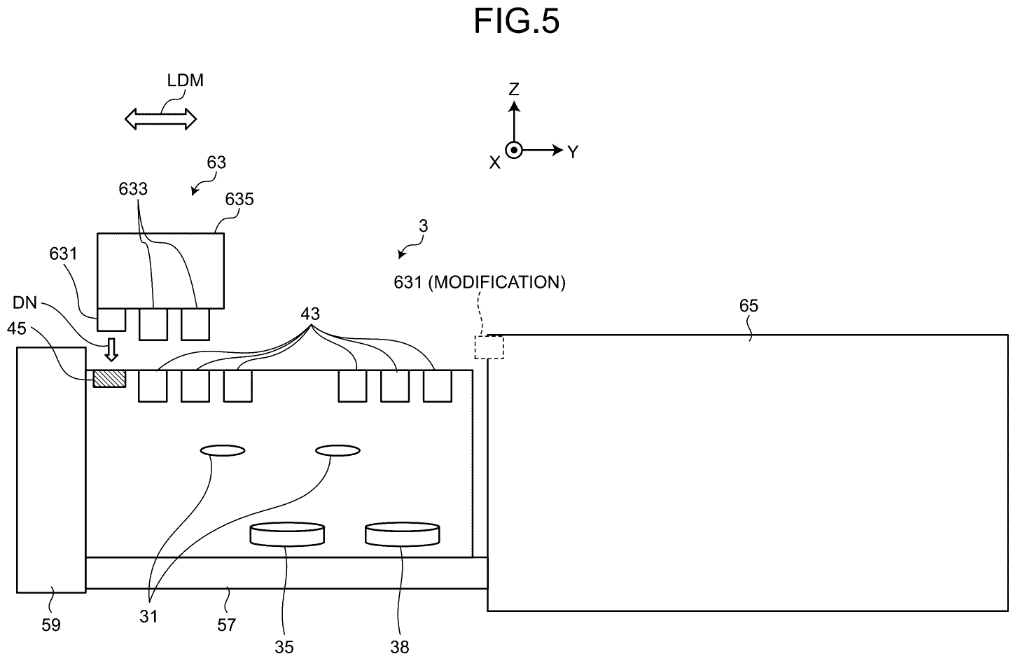

FIG. 5 is a diagram illustrating an example of a positional relationship between the level 45 and a reading apparatus 631 that reads the level 45. In FIG. 5, the reading apparatus 631 is provided together with a pressure pad 633 at a distal end of an arm 635 that movably supports the pressure pad 633. An installation position of the reading apparatus 631 is not limited to the distal end of the arm 635 in the liquid feeding apparatus 63, and for example, as illustrated in FIG. 5, the reading apparatus 631 can be installed at any position as long as the position is close to the cartridge 3 when the cartridge 3 is inserted into the sample processing apparatus 5, such as an end portion of the incubator 65 or an end portion of the cooling apparatus 59 as a modification. Hereinafter, for the sake of detailed explanation, the reading apparatus 631 will be described as being installed at the distal end of the liquid feeding apparatus 63.

When the cartridge 3 is set in the sample processing apparatus 5 and an apparatus operation instruction is input via the input interface 51, the reading apparatus 631 performs a reading operation on the level 45. In the reading operation, for example, first, under the control of the control function 673, the liquid feeding apparatus 63 is moved by the movement mechanism in the liquid feeding apparatus 63 such that a pressure pad connection portion 43 connected to the storage container 30 is positioned immediately below the pressure pad 633 and the reading apparatus 631 is positioned immediately above the level 45 (LDM). Then, when the pressure pad 633 is lowered (DN) to the pressure pad connection portion 43 by the movement mechanism, the reading apparatus 631 reads the horizontal state of the level 45 from the level 45. For example, the reading apparatus 631 reads the horizontal state from the level 45 when a distance to the level 45 becomes equal to or less than a predetermined distance. The reading apparatus 631 outputs a result (horizontal state) read from the level 45 to the processing circuitry 67. The reading apparatus 631 corresponds to a reading unit. The reading apparatus 631 may perform the reading operation also when contact between the operator OP and the cartridge 3 is assumed, for example, when the reagent or the like is set.

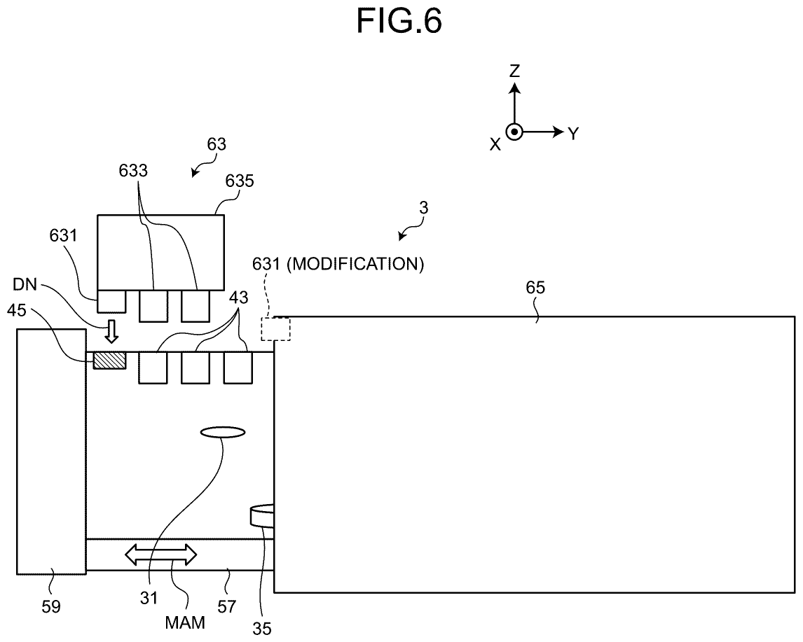

FIG. 5 illustrates a mode in which the liquid feeding apparatus 63 moves with respect to the cartridge 3 to read the level 45, but the cartridge may be moved by the conveyance apparatus 57 to read the level 45. FIG. 6 is a diagram illustrating an example of movement MDM of the cartridge 3 by the conveyance apparatus 57. At this time, in the reading operation, the cartridge 3 is moved in the Y direction together with the cooling apparatus 59. Both the liquid feeding apparatus 63 and the cartridge 3 may be moved in the reading operation or the like.

The incubator 65 provides an environment suitable for culture in the culture container 38. For example, the incubator 65 provides a carbon dioxide concentration, an oxygen concentration, a temperature, and humidity suitable for culture under the control of the control function 673. As a result, the incubator 65 adjusts the environment in the cartridge. Since a known incubator is applicable as the incubator 65, a description thereof will be omitted.

The processing circuitry 67 controls the entire operation of the sample processing apparatus 5 according to the sample processing information output from the input interface 51 or the memory 55. For example, the processing circuitry 67 includes, as hardware resources, a processor such as a central processing unit (CPU), a micro processing unit (MPU), or a graphics processing unit (GPU), and a memory such as a read only memory (ROM) or a RAM.

In addition, the processing circuitry 67 may be implemented by an application specific integrated circuit (ASIC), a field programmable gate array (FPGA), another complex programmable logic device (CPLD), a simple programmable logic device (SPLD), or the like.

The processing circuitry 67 has, for example, the information acquisition function 671, the control function 673, and the detection function 675. Each of the information acquisition function 671, the control function 673, and the detection function 675 is stored in the memory 55 in the form of a program executable by a computer. The processing circuitry 67 executes the information acquisition function 671, the control function 673, and the detection function 675 by a processor that executes a program loaded in a memory of the processing circuitry 67 itself.

That is, the processing circuitry 67 corresponds to a processor that implements a function corresponding to each program by reading and executing the program from the memory 55. In other words, the processing circuitry 67 in a state of reading each program has a function corresponding to the read program. The information acquisition function 671, the control function 673, and the detection function 675 are not limited to being implemented by single processing circuitry. Processing circuitry may be implemented by combining a plurality of independent processors and each processor may execute a program to implement each of the information acquisition function 671, the control function 673, and the detection function 675. The processing circuitry 67 that implements the information acquisition function 671, the control function 673, and the detection function 675 is an example of an information acquisition unit, a control unit, and a detection unit.

The processing circuitry 67 acquires the sample processing information output from the input interface 51 by the information acquisition function 671. The information acquisition function 671 stores the acquired sample processing information in the memory 55.

The processing circuitry 67 controls the entire sample processing apparatus 5 by the control function 673. For example, the control function 673 controls the conveyance apparatus 57, the cooling apparatus 59, the valve driving apparatus 61, the liquid feeding apparatus 63, and the incubator 65 according to the sample processing information. For example, when the cartridge 3 is inserted into the sample processing apparatus 5, the control function 673 controls at least one of the conveyance apparatus 57 and the liquid feeding apparatus 63 so as to arrange the cartridge 3 at a position related to suction and discharge of the sample according to the sample processing information. At this time, the control function 673 controls the cooling apparatus 59 to cool the cartridge 3.

The processing circuitry 67 controls the valve driving apparatus 61 according to the sample processing information by the control function 673. Specifically, the control function 673 moves a position of the valve driving apparatus 61 with respect to the cartridge 3 according to the sample processing information. Next, the control function 673 controls the valve driving apparatus 61 according to the sample processing information to drive the valve 41 provided in the cartridge 3.

The processing circuitry 67 controls the liquid feeding apparatus 63 according to the sample processing information by the control function 673. For example, the control function 673 controls movement of the liquid feeding apparatus 63 such that the storage container 30 is positioned immediately below the pressure pad 633 at the distal end of the liquid feeding apparatus 63. Next, the control function 673 controls the liquid feeding apparatus 63 so as to bring the pressure pad 633 into close contact with the pressure chamber connected to an upper end of the storage container 30. Subsequently, the control function 673 controls the liquid feeding apparatus 63 to suck or compress air via the pressure pad 633.

The processing circuitry 67 controls the incubator 65 according to the sample processing information by the control function 673. For example, when the reagent and the sample are fed to the culture container 38, the control function 673 controls the incubator 65 to implement the environment suitable for culture in the culture container 38. The control of the incubator 65 may be performed from a time point when the cartridge 3 is inserted into the sample processing apparatus 5.

The processing circuitry 67 detects the inclination of the cartridge 3 with respect to the horizontal plane and/or the apparatus body of the sample processing apparatus 5 by the detection function 675. For example, the detection function 675 detects the inclination of the cartridge 3 based on a reading result of the reading apparatus 631. Specifically, the detection function 675 determines the inclination of the cartridge 3 based on a result of reading the level 45 (for example, data such as the position of the bubble in the level 45 and an elevation angle or depression angle according to the level 45).

For example, if the result of reading the level 45 is equal to or less than a predetermined threshold, the detection function 675 detects (determines) that the cartridge 3 is not inclined. If the result of reading the level 45 exceeds the predetermined threshold, the detection function 675 detects (determines) that the cartridge 3 is inclined. Other known methods may be used to determine the inclination of the cartridge 3 based on the result of reading the level 45. The detection function 675 outputs the inclination of the cartridge 3 to the output interface 53.

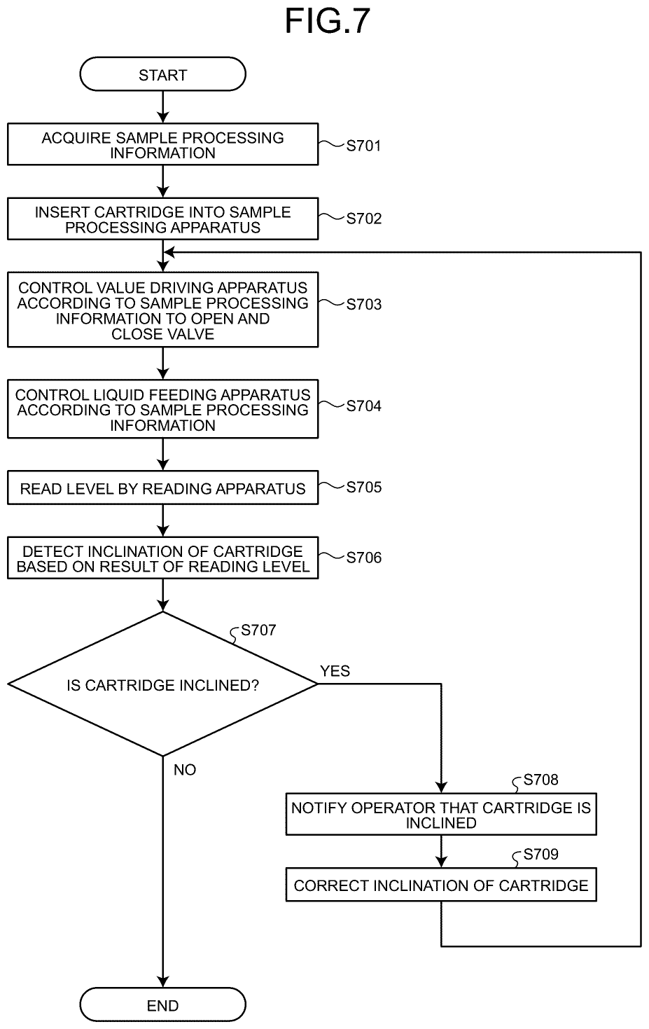

Each component of the sample processing system 1 has been described above. Hereinafter, a procedure of control processing for detecting the inclination of the cartridge 3 (hereinafter, referred to as cartridge inclination detection processing) by the sample processing apparatus 5 will be described. A method of implementing the cartridge inclination detection processing may be referred to as an inclination detection method. FIG. 7 is a flowchart illustrating an example of the procedure of the cartridge inclination detection processing. It is assumed that the sample and the reagent are set in advance in the cartridge 3 before the cartridge inclination detection processing is performed. In addition, it is assumed that the sample processing information is also set in advance before the cartridge inclination detection processing is performed.

### Cartridge Inclination Detection Processing

### Step S701

The processing circuitry 67 acquires the sample processing information by the information acquisition function 671. The information acquisition function 671 may acquire the sample processing information from each server apparatus or the like via the network. The information acquisition function 671 stores the acquired sample processing information in the memory 55.

### Step S702

The cartridge 3 is inserted into the sample processing apparatus 5 by the operation of the operator OP. At this time, the cartridge 3 is supported by the conveyance apparatus 57.

### Step S703

The processing circuitry 67 controls the conveyance apparatus 57, the cooling apparatus 59, and the like by the control function 673. In addition, the control function 673 controls the valve driving apparatus 61 according to the sample processing information. The valve driving apparatus 61 drives opening and closing of the valve 41 from the outside of the cartridge 3 by magnetic interaction with the first magnet or the magnetic body under the control of the control function 673.

### Step S704

The processing circuitry 67 controls the liquid feeding apparatus 63 according to the sample processing information by the control function 673. The liquid feeding apparatus 63 sucks and discharges the sample and/or the reagent under the control of the control function 673.

### Step S705

When the arm 635 is lowered according to the control of the liquid feeding apparatus 63 by the control function 673, the reading apparatus 631 reads the horizontal state from the level 45. The reading apparatus 631 outputs the result of reading the level 45 to the processing circuitry 67.

### Step S706

The processing circuitry 67 detects the inclination of the cartridge 3 with respect to the horizontal plane and/or the apparatus body based on the reading result by the detection function 675. The detection function 675 outputs the inclination of the cartridge 3 to the output interface 53.

### Step S707

If the inclination of the cartridge 3 is detected (Yes in step S707), the processing in and after step S708 is performed. If the inclination of the cartridge 3 is not detected (No in step S707), the cartridge inclination detection processing ends. At this time, the output interface 53 may notify the operator OP that the inclination of the cartridge 3 is not detected (for example, a deviation amount (angle) from the horizontal plane or the cartridge 3 is horizontal) by using a display, a microphone, or the like.

### Step S708

The processing circuitry 67 controls the output interface 53 by the control function 673. As a result, the output interface 53 notifies the operator OP that the cartridge 3 is inclined by using a display, a microphone, or the like. At this time, various operations by the valve driving apparatus 61, the liquid feeding apparatus 63, and the like are stopped.

### Step S709

The inclination of the cartridge 23 is corrected by the operation of the operator OP. For example, the cartridge 3 is taken out from the sample processing apparatus 5 in response to an instruction received from the operator OP via the input interface 51. The cartridge 3 is inserted into the sample processing apparatus 5 again by the operator OP. As a result, the correction of the inclination of the cartridge 23 is completed. The inclination of the cartridge 23 may be corrected by operating each of a plurality of actuators (tilting mechanisms) under the control of the control function 673. The tilting mechanism is provided, for example, on a stage installed immediately above the conveyance apparatus 57 or the conveyance apparatus 57 itself. The stage adjusts the inclination of the cartridge 3 according to the control of the tilting mechanism by the control function 673.

The sample processing apparatus 5 according to the embodiment described above includes the apparatus body in which the cartridge 3 for internally performing processing on the cell is installed, and which controls liquid feeding in the cartridge and/or adjusts the environment in the cartridge, and detects the inclination of the cartridge 3 with respect to the horizontal plane and/or the apparatus body. In addition, the cartridge 3 in the sample processing apparatus 5 according to the embodiment includes the level 45 that reflects the degree of horizontality of the cartridge 3, and the apparatus body of the sample processing apparatus 5 reads the level 45 and detects the inclination of the cartridge 3 based on the result of reading the level 45.

Therefore, with the sample processing apparatus 5 according to the embodiment, the reading apparatus 631 is mounted on the liquid feeding apparatus 63, and an operation axis of the liquid feeding apparatus 63 in a connection operation between the pressure pad connection portion 43 and the pressure pad 633 at the time of liquid feeding can also be used for the reading operation, so that the cartridge inclination detection processing can be implemented without increasing the number of operation axes related to the reading operation. As a result, with the sample processing apparatus 5 according to the embodiment, it is possible to notify the operator OP or the like of the inclination of the cartridge 3 with respect to the horizontal plane and/or the apparatus body. Therefore, with the sample processing apparatus 5 according to the embodiment, it is possible to appropriately process the sample such as cell suspension by correcting the inclination of the cartridge 3, that is, by maintaining the cartridge 3 in the horizontal state.

### First Modification

A first modification of the present embodiment is to install the level 45 on the filter 31, the culture container 38, the expansion culture container 35, and/or the like in the cartridge 3 via a jig.

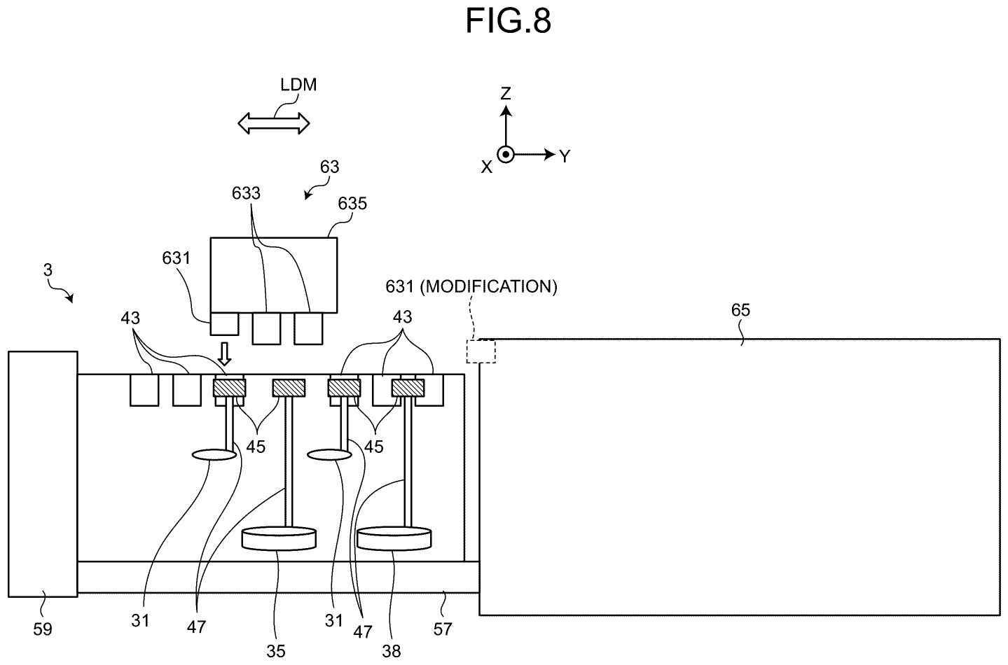

FIG. 8 is a diagram illustrating an example of a positional relationship between the level 45 and the reading apparatus 631 that reads the level 45 in the first modification. Although a plurality of levels 45 are illustrated in FIG. 8, the level 45 may be implemented by one level. As illustrated in FIG. 8, the filter 31, the culture container 38, and the expansion culture container 35 are horizontally fixed to the cartridge 3 via fixtures. A jig 47 is connected to each of fixtures related to the filter 31, the culture container 38, and the expansion culture container 35. The level 45 is arranged, for example, on the upper surface of the cartridge 3 via the jig 47. Since cartridge inclination detection processing in the present modification is similar to that of the embodiment, a description thereof will be omitted.

With the sample processing apparatus 5 according to the present modification, it is possible to directly detect the inclination of the culture container 38 and the filter 31 with respect to the horizontal plane and/or the apparatus body. Therefore, according to the present modification, the sample such as cell suspension can be more appropriately processed. Since other effects in the present modification are the same as those of the embodiment, a description thereof will be omitted.

### Second Modification

In a second modification of the present embodiment, the apparatus body of the sample processing apparatus 5 includes a contact portion that can come into contact with the cartridge 3. In the second modification, the level in the cartridge 3 is unnecessary. The processing circuitry 67 detects the inclination of the cartridge 3 based on a contact result when the contact portion comes into contact with the cartridge 3 by the detection function 675. Hereinafter, in order to make the description specific, the contact portion will be described as a contact apparatus that comes into contact with the cartridge 3 at a variable angle. At this time, the contact apparatus is implemented by, for example, a level or an angle meter. In the present modification, the mounting of the level 45 on the cartridge 3 and the reading apparatus 631 are unnecessary.

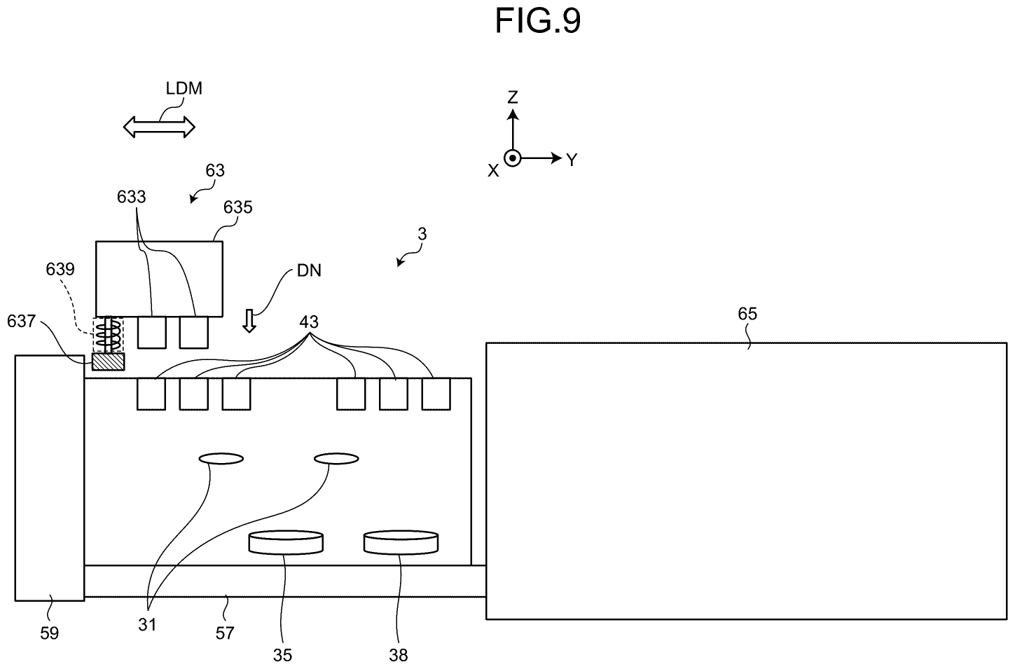

FIG. 9 is a diagram illustrating an example of a positional relationship between a contact apparatus 637 and the cartridge 3. As illustrated in FIG. 9, the contact apparatus 637 is supported by the arm 635 in the liquid feeding apparatus 63 via a damper (shock absorber) 639. The damper 639 is used to absorb impact and vibration on the contact apparatus 637 and the cartridge 3 when the contact apparatus 637 comes into contact with the cartridge 3. In FIG. 9, the damper 639 is illustrated as, for example, a spring and a cylinder, but a known damper can be applied. The contact apparatus 637 and the damper 639 are connected via a ball joint. Therefore, the contact apparatus 637 can smoothly come into contact with the cartridge 3.

An installation position of the contact apparatus 637 is not limited to the distal end of the arm 635 in the liquid feeding apparatus 63 as illustrated in FIG. 9, and as a modification, the contact apparatus 637 can be installed at any position as long as the position is a position where the contact apparatus 637 can come into contact with the cartridge 3 when the cartridge 3 is inserted into the sample processing apparatus 5, such as the end portion of the incubator 65 or the end portion of the cooling apparatus 59. Hereinafter, for the sake of detailed explanation, the contact apparatus 637 will be described as being installed at the distal end of the liquid feeding apparatus 63.

The liquid feeding apparatus 63 is moved by the movement mechanism in the liquid feeding apparatus 63 such that the pressure pad connection portion 43 connected to the storage container 30 is positioned immediately below the pressure pad 633 (LDM) under the control of the control function 673. Then, when the pressure pad 633 is lowered (DN) by the movement mechanism to the pressure pad connection portion 43, the contact apparatus 637 comes into contact with the cartridge 3 at a variable angle by the ball joint. At this time, the contact apparatus 637 outputs a contact result when the contact apparatus 637 comes into contact with the cartridge 3, for example, an inclination (data of a position of a bubble in a bubble tube in a case where the contact apparatus 637 is a level and data of an angle in a case where the contact apparatus 637 is an angle meter) of the contact apparatus 637 to the processing circuitry 67.

The processing circuitry 67 detects the inclination of the cartridge 3 based on the contact result of the contact apparatus 637 by the detection function 675. For example, the detection function 675 detects the inclination of the cartridge 3 based on the inclination of the contact apparatus 637 when the contact apparatus 637 is brought into contact with the cartridge 3. Specifically, the detection function 675 determines the inclination of the cartridge 3 based on the contact result (data such as the position of the bubble in the level or the elevation or depression angle according to the angle meter) of the contact apparatus 637. Since contents of the determination are the same as those of the embodiment, a description thereof will be omitted.

A procedure of the cartridge inclination detection processing in the present modification is similar to that of the cartridge inclination detection processing illustrated in FIG. 7 except for step S705 and step S706. Hereinafter, processing of step S705 and processing of step S706 in the present modification will be described.

### Step S705

When the arm 635 is lowered according to the control of the liquid feeding apparatus 63 by the control function 673, the contact apparatus 637 comes into contact with the cartridge 3. The contact apparatus 637 outputs the contact result to the processing circuitry 67.

### Step S706

The processing circuitry 67 detects the inclination of the cartridge 3 with respect to the horizontal plane and/or the apparatus body based on the contact result by the detection function 675. The detection function 675 outputs the inclination of the cartridge 3 to the output interface 53.

The apparatus body of the sample processing apparatus 5 according to the present modification further includes the contact portion that can come into contact with the cartridge 3, and detects the inclination of the cartridge 3 based on the contact result when the contact portion 637 comes into contact with the cartridge 3. For example, the contact portion 637 in the sample processing apparatus 5 according to the second modification comes into contact with the cartridge 3 at a variable angle, and detects the inclination of the cartridge 3 based on the inclination of the contact portion 637 when the contact portion 637 is brought into contact with the cartridge 3. Since other effects in the present modification are the same as those of the embodiment, a description thereof will be omitted.

### Third Modification

In a third modification of the present embodiment, the apparatus body of the sample processing apparatus 5 includes the contact portion that can come into contact with the cartridge 3. In the third modification, the level 45 is unnecessary. The contact portion is provided on the pressure pad 633. At this time, the processing circuitry 67 detects the inclination of the cartridge 3 based on the contact result when the contact portion comes into contact with the cartridge 3 by the detection function 675.

Hereinafter, in order to make the description specific, the contact portion will be described as a contact sensor that is lowered from each of a plurality of positions on the horizontal plane and comes into contact with the cartridge 3. The contact sensor is, for example, a pressure detection type sensor, and is mounted on the pressure pad 633. The contact portion is not limited to the contact sensor, and various contact sensors of known types can be applied. In addition, the determination when the contact portion comes into contact with the cartridge 3 is not limited to a response (ON state) of the contact sensor, and may be performed by, for example, a change in load (a change in current) of a motor that causes the arm 635 to perform processing. At this time, the contact sensor is unnecessary, and the contact portion corresponds to the pressure pad.

As an application example of the present modification, laser ranging, light detection and ranging (LiDAR), and the like may be performed at a plurality of positions on the same horizontal plane. At this time, the processing circuitry 67 detects the inclination of the cartridge 3 by detecting distances between the upper surface of the cartridge 3 and the plurality of positions on the horizontal plane by the detection function 675.

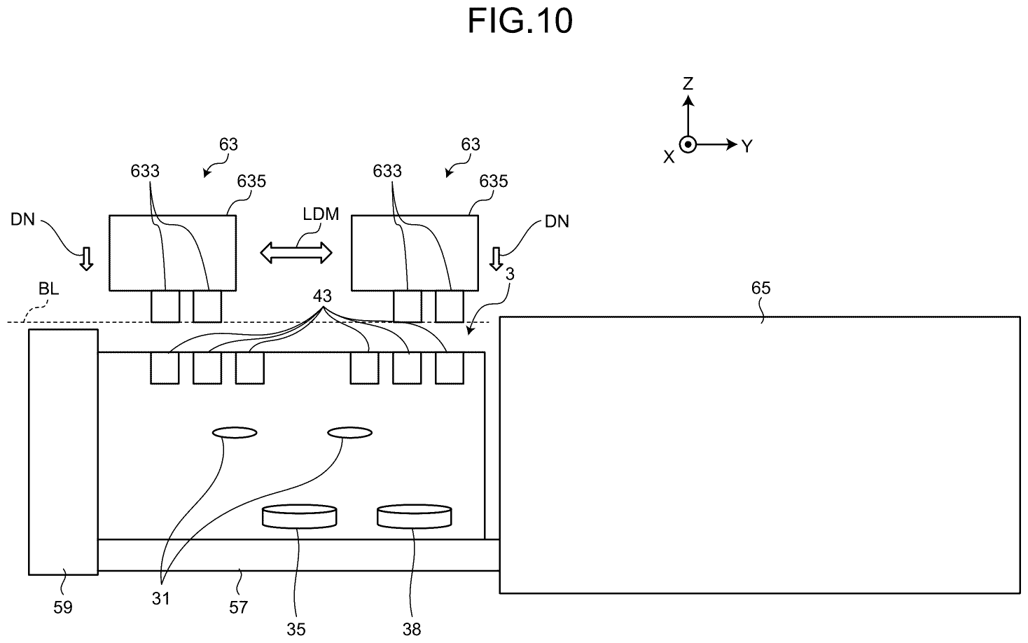

FIG. 10 is a diagram illustrating an example of a positional relationship between the arm 635 and the cartridge 3 at two positions. A dotted line BL illustrated in FIG. 10 corresponds to a reference line (horizontal plane) for detecting the inclination of the cartridge 3. As illustrated in FIG. 10, the pressure pads 633 are lowered toward the pressure pad connection portions 43 at two positions.

The liquid feeding apparatus 63 is moved by the movement mechanism in the liquid feeding apparatus 63 such that the pressure pad connection portion 43 is positioned immediately below the pressure pad 633 (LDM) under the control of the control function 673. Then, when the pressure pad 633 is lowered (DN) by the movement mechanism to the pressure pad connection portion 43, the pressure pad 633 detects contact between the cartridge 3 and the pressure pad 633 by the contact sensor. As a result, the contact portion outputs a time when the pressure pad 633 comes into contact with the cartridge 3 (hereinafter, referred to as a contact time) to the processing circuitry 67. The contact time is detected at each of the plurality of positions (two points in FIG. 10 (hereinafter, referred to as a first position and a second position)). Hereinafter, the contact time at the first position is referred to as a first time, and the contact time at the second position is referred to as a second time.

The processing circuitry 67 detects the inclination of the cartridge 3 based on a length from the contact portion at each of the plurality of positions to the cartridge 3 by the detection function 675. Specifically, the detection function 675 calculates a distance (hereinafter, referred to as a first distance) from the reference line by multiplying a difference between a lowering start time (hereinafter, referred to as a first lowering start time) of the pressure pad 633 at the first position and the first time by a lowering speed of the pressure pad 633. The detection function 675 may calculate the first distance by using an output value of a rotary encoder in a motor related to the lowering of the pressure pad 633 during a time from the first lowering start time to the first time.

In addition, the detection function 675 calculates a distance (hereinafter, referred to as a second distance) from the reference line by multiplying a difference between a lowering start time (hereinafter, referred to as a second lowering start time) of the pressure pad 633 at the second position and the second time by the lowering speed of the pressure pad 633. The detection function 675 may calculate the second distance by using an output value of the rotary encoder in the motor related to the lowering of the pressure pad 633 during a time from the second lowering start time to the second time.

The processing circuitry 67 detects (determines) the inclination of the cartridge 3 by using the first distance and the second distance by the detection function 675. Since contents of the determination are the same as those of the embodiment, a description thereof will be omitted.

A procedure of the cartridge inclination detection processing in the present modification is similar to that of the cartridge inclination detection processing illustrated in FIG. 7 except for step S705 and step S706. Hereinafter, processing of step S705 and processing of step S706 in the present modification will be described.

### Step S705

When the arm 635 is lowered at the first position according to the control of the liquid feeding apparatus 63 by the control function 673, the pressure pad 633 comes into contact with the cartridge 3. At this time, the contact portion outputs the first time to the processing circuitry 67. In addition, when the arm 635 is lowered at the second position according to the control of the liquid feeding apparatus 63 by the control function 673, the pressure pad 633 comes into contact with the cartridge 3. At this time, the contact portion outputs the second time to the processing circuitry 67.

### Step S706

The processing circuitry 67 calculates the first distance based on the first lowering start time and the first time by the detection function 675. In addition, the detection function 675 calculates the second distance based on the second lowering start time and the second time. Next, the detection function 675 detects (determines) the inclination of the cartridge 3 based on the first distance and the second distance. That is, the detection function 675 detects the inclination of the cartridge 3 based on the length from the contact portion at each of the plurality of positions to the cartridge 3.

The contact portion in the sample processing apparatus 5 according to the present modification is lowered from each of the plurality of positions on the horizontal plane and comes into contact with the cartridge 3, and the sample processing apparatus 5 detects the inclination of the cartridge 3 based on the length from the contact portion at each of the plurality of positions to the cartridge 3. Since other effects in the present modification are the same as those of the embodiment, a description thereof will be omitted.

### Fourth Modification

In a fourth modification of the embodiment, a plurality of acquisition units that acquire information regarding the inclination of the cartridge 3 from below the cartridge 3 placed on the conveyance apparatus 57 are mounted on the conveyance apparatus 57, and the inclination of the cartridge 3 is detected based on output results output from the plurality of acquisition units. In the fourth modification, the level 45 is unnecessary. The plurality of acquisition units are, for example, a plurality of distance sensors or a plurality of contact sensors. The distance sensor is implemented by, for example, a known sensor such as laser ranging or LiDAR. In addition, since the contact sensor can be implemented by a known form such as a switch, a description thereof will be omitted.

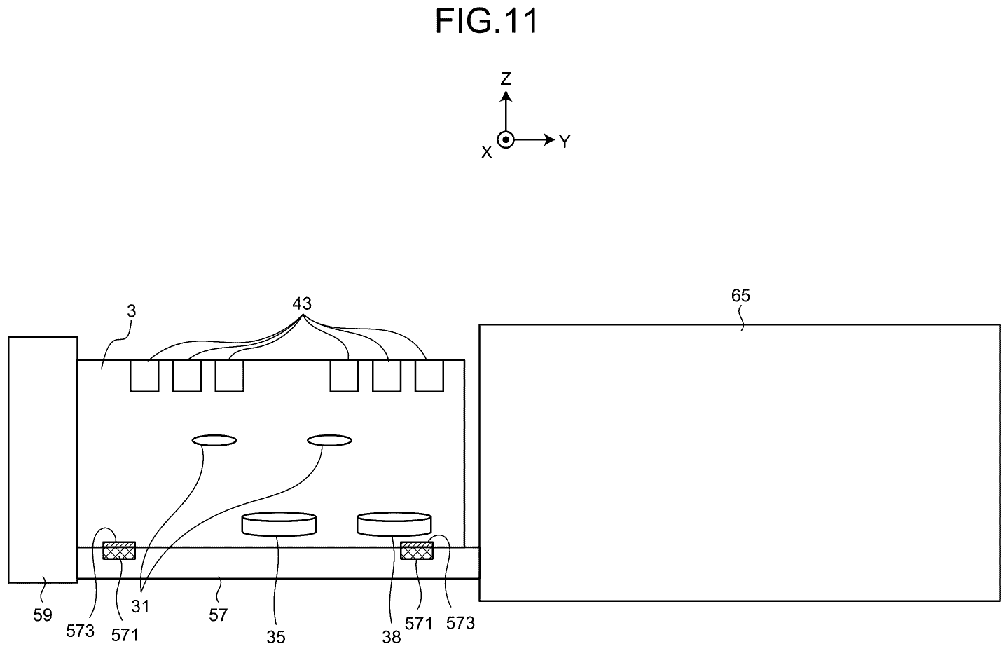

FIG. 11 is a diagram illustrating an example of a positional relationship between a plurality of sensors 571 and the cartridge 3. As illustrated in FIG. 11, the plurality of sensors 571 are arranged in recesses that are opend upward in the vertical direction. The plurality of sensors 571 may be arranged immediately below holes provided in the conveyance apparatus 57. As illustrated in FIG. 11, the cartridge 3 includes a detection target portion 573 corresponding to a target to be detected by each of the plurality of sensors 571. The detection target portion 573 has, for example, a shape that is fitted into the recess or hole provided in the conveyance apparatus 57. Hereinafter, for the sake of detailed explanation, it is assumed that the plurality of sensors 571 are two sensors arranged at two positions as illustrated in FIG. 11.

In a case where the two sensors 571 are distance sensors, the two distance sensors measure a distance to a bottom surface of the cartridge 3 from below the cartridge 3. The plurality of distance sensors output the distance to the processing circuitry 67. In a case where the two sensors 571 are contact sensors, the contact sensors determine whether or not there is contact with the bottom surface (the detection target portion 573) of the cartridge 3. Each of the contact sensors outputs whether or not there is contact (ON indicates contact and OFF indicates non-contact) to the processing circuitry 67.

The processing circuitry 67 detects the inclination of the cartridge 3 based on the output results output from the plurality of acquisition units 571 by the detection function 675. For example, in a case where the plurality of acquisition units 571 are two distance sensors, the detection function 675 determines whether or not both of two distances at two points exceed a threshold (hereinafter, referred to as a distance threshold). In a case where the two distances at the two points both exceed the distance threshold, the detection function 675 detects that the cartridge 3 is floating (hereinafter, referred to as a floating error). In addition, in a case where one of the two distances at the two points exceeds the distance threshold, the detection function 675 detects that the cartridge 3 is inclined (hereinafter, referred to as an inclination error). In addition, in a case where both of the two distances at the two points are equal to or less than the distance threshold, the detection function 675 detects that the cartridge 3 is in the horizontal state (hereinafter, referred to as no abnormality).

In addition, for example, in a case where the plurality of acquisition units 571 are two contact sensors, the detection function 675 determines whether or not the cartridge 3 is inclined by a combination of ON/OFF at two points. In a case where both the outputs from the two contact sensors are OFF (OFF and OFF), the detection function 675 detects the floating error. In a case where the outputs from the two contact sensors are ON and OFF (ON and OFF), the detection function 675 detects the inclination error. In a case where the outputs from the two contact sensors are both ON (ON and ON), the detection function 675 detects no abnormality. The detection function 675 outputs “floating error”, “inclination error”, or “no abnormality” to the output interface 53.

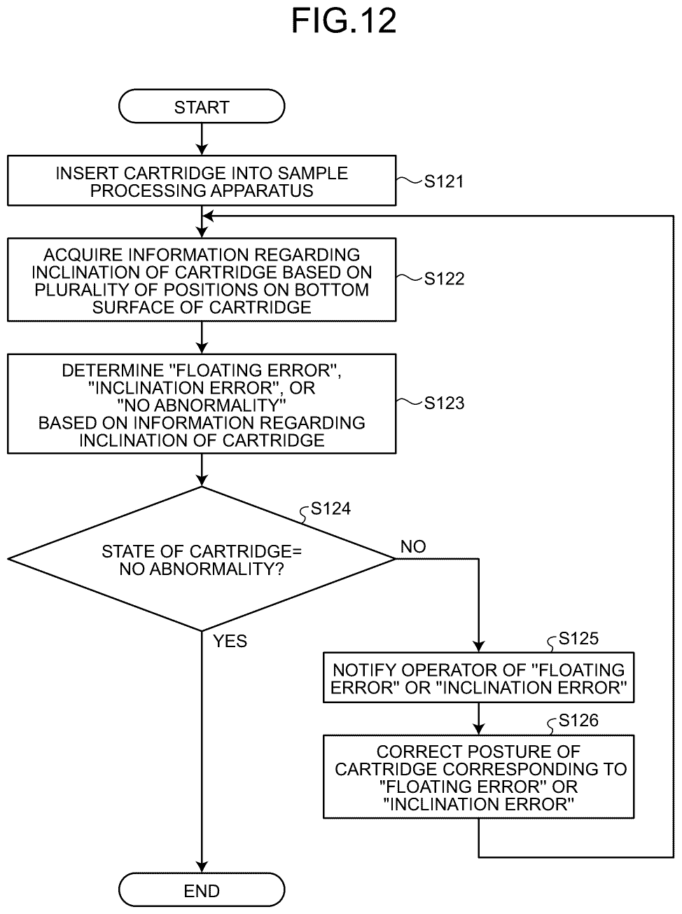

The cartridge inclination detection processing in the present modification will be described with reference to FIG. 12. FIG. 12 is a flowchart illustrating an example of a procedure of the cartridge inclination detection processing according to the fourth modification.

### Cartridge Inclination Detection Processing

### Step S121

The cartridge 3 is inserted into the sample processing apparatus 5 by the operation of the operator OP. At this time, the cartridge 3 is supported by the conveyance apparatus 57.

### Step S122

The plurality of acquisition units 571 acquire information regarding the inclination of the cartridge 3 based on a plurality of positions on the bottom surface of the cartridge 3. The plurality of acquisition units 571 output the information regarding the inclination of the cartridge 3 to the processing circuitry 67. In a case where the plurality of acquisition units 571 are a plurality of distance sensors, the plurality of distance sensors acquire a plurality of distances corresponding to the plurality of positions as the information regarding the inclination of the cartridge 3. Furthermore, in a case where the plurality of acquisition units 571 are a plurality of contact sensors, the plurality of contact sensors acquire the presence or absence (ON or OFF) of contact corresponding to the plurality of positions as the information regarding the inclination of the cartridge 3.

### Step S123

The processing circuitry 67 determines (detects) “floating error”, “inclination error”, or “no abnormality” based on the information regarding the inclination of the cartridge 3. The detection function 675 outputs the determination result (detection result) to the output interface 53.

### Step S124

If the state of the cartridge 3 is “no abnormality” (Yes in step S124), the cartridge inclination detection processing ends. Next, various types of processing are performed on the sample (cell) in the cartridge 3. At this time, the output interface 53 may notify the operator OP that the inclination of the cartridge 3 is not detected (for example, “no abnormality”) by using a display, a microphone, or the like. If the state of the cartridge 3 is not “no abnormality” (No in step S124), the processing in and after step S125 is performed.

### Step S125

The processing circuitry 67 controls the output interface 53 by the control function 673. As a result, the output interface 53 notifies the operator OP of the determination result such as the “floating error” or the “inclination error” through a display, a microphone, or the like.

### Step S126

A posture of the cartridge 3 corresponding to the determination result such as “floating error” or “inclination error” is corrected by the operation of the operator OP. For example, the cartridge 3 is taken out from the sample processing apparatus 5 in response to an instruction received from the operator OP via the input interface 51. Next, the cartridge 3 is inserted into the sample processing apparatus 5 again by the operator OP. As a result, the correction of the determination result such as “floating error” or “inclination error” is completed.

The correction of the posture of the cartridge 3 may be performed by operating each of the plurality of actuators (tilting mechanisms) under the control of the control function 673. The tilting mechanism is provided, for example, on a stage installed immediately above the conveyance apparatus 57 or the conveyance apparatus 57 itself. The stage adjusts the inclination of the cartridge 3 according to the control of the tilting mechanism by the control function 673.

The conveyance unit in the sample processing apparatus 5 according to the present modification, on which the cartridge 3 can be placed, conveys the cartridge 3 in the horizontal direction, and includes the plurality of acquisition units 571 that acquire the information regarding the inclination of the cartridge 3 based on the plurality of positions on the bottom surface of the cartridge 3, and the sample processing apparatus 5 detects the inclination of the cartridge 3 based on the output results output from the plurality of acquisition units 571. The plurality of acquisition units 571 in the sample processing apparatus 5 according to the present modification are a plurality of distance sensors or a plurality of contact sensors, the plurality of distance sensors measure the distance to the bottom surface of the cartridge 3 from below the cartridge 3, and the plurality of contact sensors determine whether or not there is contact with the bottom surface of the cartridge 3. Since the effects of the present modification are the same as those of the embodiment, a description thereof will be omitted.

In a case where the technical features in the present embodiment are implemented by the sample processing system 1, the sample processing system 1 includes the cartridge 3 for internally performing processing on the cell, and the sample processing apparatus 5 in which the cartridge 3 is installed and which controls liquid feeding in the cartridge and/or adjusts the environment in the cartridge, and the sample processing apparatus 5 includes the detection unit that detects the inclination of the cartridge 3 with respect to the horizontal plane and/or the sample processing apparatus 5. Since the procedure and effect of the cartridge inclination detection processing in the sample processing system 1 are similar to those of the embodiment, a description thereof will be omitted.

In a case where the technical features in the present embodiment are implemented by the cartridge 3, the cartridge 3 includes a cartridge body for internally performing processing on the cell, the cartridge body including the culture container 38 for cell culture and the filter 31 for collecting the target cell related to cell culture, and the level 45 that reflects the degree of horizontality of the cartridge body, and the culture container 38 and the filter 31 are fixed to a housing of the cartridge body. Since the procedure and effect of the cartridge inclination detection processing in the cartridge 3 are similar to those of the embodiment, a description thereof will be omitted.

According to at least one embodiment, at least one modification, and the like described above, it is possible to implement detection of the inclination of the cartridge 3 for the cartridge 3 for internally performing processing on the cell.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A sample processing apparatus comprising:
an apparatus body in which a cartridge for internally performing processing on a cell is installed and which controls liquid feeding in the cartridge and/or adjusts an environment in the cartridge,
wherein the apparatus body includes processing circuitry configured to detect an inclination of the cartridge with respect to a horizontal plane and/or the apparatus body.

2. The sample processing apparatus according to claim 1, wherein
the cartridge includes a level that measures a horizontal state of the cartridge,
the apparatus body further includes a reading apparatus that reads the horizontal state from the level, and
the processing circuitry is configured to detect the inclination of the cartridge based on a reading result of the reading apparatus.

3. The sample processing apparatus according to claim 1, wherein
the apparatus body further includes a contact apparatus that is configured to come into contact with the cartridge, and
the processing circuitry is configured to detect the inclination of the cartridge based on a contact result when the contact apparatus comes into contact with the cartridge.

4. The sample processing apparatus according to claim 3, wherein
the contact apparatus comes into contact with the cartridge at a variable angle, and
the processing circuitry is configured to detect the inclination of the cartridge based on an inclination of the contact portion when the contact portion is brought into contact with the cartridge.

5. The sample processing apparatus according to claim 3, wherein
the contact apparatus is lowered from each of a plurality of positions on a horizontal plane and comes into contact with the cartridge, and
the processing circuitry is configured to detect the inclination of the cartridge based on a length from the contact portion at each of the plurality of positions to the cartridge.

6. The sample processing apparatus according to claim 1, wherein the apparatus body further includes a conveyance apparatus on which the cartridge is placeable and which conveys the cartridge in a horizontal direction,
the conveyance apparatus includes a plurality of sensors that acquire information regarding the inclination of the cartridge based on a plurality of positions on a bottom surface of the cartridge, and
the processing circuitry is configured to detect the inclination of the cartridge based on output results output from the plurality of sensors.

7. The sample processing apparatus according to claim 6, wherein
the plurality of sensors are a plurality of distance sensors or a plurality of contact sensors,
the plurality of distance sensors measure a distance to the bottom surface of the cartridge from below the cartridge, and
the plurality of contact sensors determine whether or not there is contact with the bottom surface of the cartridge.

8. The sample processing apparatus according to claim 1, wherein the cartridge includes a culture container for cell culture, and
the culture container is fixed to the cartridge.

9. The sample processing apparatus according to claim 1, wherein the cartridge includes a filter for collecting a target cell, and
the filter is fixed to the cartridge.

10. A sample processing system comprising:
a cartridge for internally performing processing on a cell; and
a sample processing apparatus in which the cartridge is installed and which controls liquid feeding in the cartridge and/or adjusts an environment in the cartridge,
wherein the sample processing apparatus includes processing circuitry configured to detect an inclination of the cartridge with respect to a horizontal plane and/or the sample processing apparatus.

11. A cartridge comprising:
a cartridge body for internally performing processing on a cell, the cartridge body including a culture container for cell culture and a filter for collecting a target cell related to the cell culture; and
a level that reflects a degree of horizontality of the cartridge body,
wherein the culture container and the filter are fixed to a housing of the cartridge body.
